(19) United States
(12) Patent Application Publication
(10) Pub. No.: US 2026/0114858 A1
(43) Pub. Date: Apr. 30, 2026

(54) MICROINVASIVE SURGERY DEVICE

(71) Applicant: University of Basel, Basel (CH)

(72) Inventors: Conrad Heid, Basel (CH); Thomas Wegele, Garching b. (DE); Philipp Preissler, München (DE); Lukas Eggenberger, Suhr (CH); Georg Rauter, Eiken (CH)

(21) Appl. No.: 19/116,812

(22) PCT Filed: Sep. 29, 2023
(86) PCT No.: PCT/EP2023/077049

§ 371 Date: Mar. 28, 2025

(30) Foreign Application Priority Data
Sep. 29, 2022 (CH) 001131/2022

(51) Int. Cl. A61B 17/00

(52) U.S. Cl. CPC A61B 17/00234; A61B 2017/00345; A61B 2017/00393; A61B 2017/00411; A61B 2017/00464; A61B 2017/00681

(57) ABSTRACT

A microinvasive surgery device includes a body element, a tube component, a plurality of tools each having an operative portion to contact a target and a proximal portion, a magazine having a plurality of chambers each configured to receive a tool, a tool interchange mechanism, and a power supply arrangement. The tool interchange mechanism activates a tool by advancing the tool through the tube component and mounting the tool to a distal portion of the tube component, and deactivates the tool by demounting the tool and retracting the tool through the tube component. The power supply arrangement provides electricity to a proximal portion of a tool when the tool is mounted. Each tool is configured either to transmit electricity through the proximal portion to the operative portion or to electrically isolate the proximal portion so that no electrical transmission to the operative portion is possible when the tool is mounted.

### TECHNICAL FIELD

The present invention relates to a microinvasive surgery device according to the preamble of independent claim 1. Such devices comprising a body element, a tube component having a proximal portion and a distal portion, wherein the proximal portion of the tube component is connected to the body element, a plurality of tools each having an operative portion to contact a target and a proximal portion, a magazine having a plurality of chambers each configured to receive one of the plurality of tools, and a tool interchange mechanism configured to activate one of the plurality of tools by advancing the one of the plurality of tools from its chamber of the magazine through the tube component to the distal portion of the tube component and mounting the forwarded tool to the distal portion of the tube component, and to deactivate the one of the plurality of tools by demounting the tool from the distal portion of the tube component and retracting the demounted one of the tools from the distal portion of the tube component through the tube component into its chamber of the magazine, wherein the tool interchange mechanism has a selection structure to select one of the plurality of tools in the magazine to be activated, can be used for a broad variety of laparoscopic or similar microinvasive surgery operations.

### BACKGROUND ART

In modern laparoscopic surgery and similar microinvasive operations, it is often desired to use different tools within one course of operation. Therefore, microinvasive surgery devices are known which allow an interchange of the different tools. For example, laparoscopic devices having magazines with plural tools are known. Such devices may allow change of the tools by displacing the tools from the magazine to a tip of the laparoscope or, vice versa, to remove the tools from the tip and store them in the magazine.

Further, the role of tissue coagulation has been growing in laparoscopic and similar microinvasive applications over the last years. In particular, in laparoscopic surgery it is very important being able to coagulate and thereby seal vessels in tissue to be intersected. If the tissue and the vessels in the inside are not sealed before the intersection, high and often uncontrolled blood loss typically is the consequence. This may lead to intraoperative complications and to inferior post-operative outcome.

Therefore, there is a need for a system allowing an efficient application of different tools in surgical operation and to induce coagulation when desired.

### DISCLOSURE OF THE INVENTION

According to the invention this need is settled by a microinvasive surgery device as it is defined by the features of independent claim 1. Preferred embodiments are subject of the dependent claims.

In particular, the invention is a microinvasive surgery device comprising a body element, a tube component, a plurality of tools, a magazine, a tool interchange mechanism and a power supply arrangement.

The tube component has a proximal portion and a distal portion. The proximal portion of the tube component is connected to the body element. Each of the plurality of tools has an operative portion to contact a target. The magazine has a plurality of chambers each configured to receive one of the plurality of tools.

The tool interchange mechanism is configured to activate one of the plurality of tools by advancing the one of the plurality of tools from its chamber of the magazine through the tube component to the distal portion of the tube component and mounting the forwarded tool to the distal portion of the tube component. It further is configured to deactivate the one of the plurality of tools by demounting the tool from the distal portion of the tube component and retracting the demounted one of the tools from the distal portion of the tube component through the tube component into its chamber of the magazine. The tool interchange mechanism has a selection structure to select one of the plurality of tools in the magazine to be activated.

The operative portion of each of the plurality of tools typically is the portion of said tool contacting the target upon operation. Such operative portion can, e.g., be one or more cutting edges or clamping elements. Parts (all parts or some designated ones) of the operative portion intended to be electrified can be provided with electricity. The proximal portion of one of the tools typically is the portion of said tool that remains inside or at the tube component when the tool is mounted to the distal portion of the tube component.

The power supply arrangement is configured to provide electricity to the proximal portion of one of the plurality of tools, when being mounted to the tube component. Each of the plurality of tools is configured either to transmit electricity through its proximal portion to the operative portion or to electrically isolate its proximal portion so that no electrical transmission to the operative portion is possible, when being mounted to the distal portion of the tube component.

The body element can be or have a housing receiving and/or enclosing other components of the device. The tube component can be any structure having a tube or tube-like member. In particular, the tube component can be or have a hollow rod-like element. The microinvasive surgery device can be a laparoscope.

When being activated, the tools can be operated. Typically, such operation is performed by a skilled physician. Thereby, operating the tool can relate to a cutting movement, e.g., by a scissor tool, a clamping movement, e.g., by a pliers tool, or the like.

The operative portion of one of the tools is the portion of said tool contacting the target upon operation of the tool. Such operative portion can, e.g., be one or more cutting edges or clamping elements of the tool. Parts (all parts or some designated ones) of the operative portion intended to be electrified can be provided with electricity.

The tools can be configured to transmit electricity through the proximal portion to the operative portion by electrically connecting the operative portion to the proximal portion. Or, they can be configured to isolate the proximal portion of one of the tools from the power supply arrangement, e.g., by means of insulation layers arranged between the tool and the tube component or the rod when being mounted to the distal portion of the tube component.

The term “distal” as used in connection with the tube components relates to a direction towards a structure to be treated by the activated tool. For example, such structure can be a tissue inside a human or animal body. Vice versa, the term “proximal” relates to a direction opposite to distal. In particular, proximal can be a direction away from the structure to be treated by the activated tool.

The selection structure of the tool interchange mechanism can be a structure directly movable into an appropriate position by a practitioner or surgeon. Or it can be a means moving a structure to be appropriately positioned. For example, it can be a bottom causing a motor to move a structure to change the tool. Or, it can be a fully automatic configuration such as a programmable robotic implementation.

The term “mount” in connection with the tool and the distal portion of the tube component typically relates to a stationary or stable connection such that, when being mounted, the tool is blocked in position or location relative to the distal portion of the tube. In such blocked position, parts of the tool may still be moved relative to the tube component. However, the position or location of the tool relative to the distal portion of the tube component cannot be changed without demounting the tool. Further, mounting of the tool to the distal portion of the tube component can be a direct mounting or an indirect mounting via another structure. For example, an indirect mounting can be provided via a rod or bar extending through the tube component and being axially fixed relative to the tube component.

The power supply arrangement can be embodied to be connected to an internal or external energy source.

The microinvasive surgery device according to the invention enables the practitioner or surgeon to perform a broad range of steps of operations in laparoscopic or endoscopic surgery with only one device. In particular, in addition to operate various tools to the tissue to be treated, also coagulation of the tissue can be induced by means of electricity provided to the tool, if desired. In particular, it allows efficient application of different tools in surgical operation and to induce coagulation when desired.

Furthermore, by designing the tools either to electrify their operative portions or to prevent electrification of their operative portions, depending on the type of operation electricity and, thus, coagulation can selectively be involved when desired or useful. For example, provision of electricity can be prevented in tools which do not require coagulation. Thus, the invention provides a multifunctional laparoscopic or other microinvasive surgery device with integrated, intelligent coagulation functions.

Preferably, the power supply arrangement comprises a first pole line configured to provide electricity from the body element to the proximal portion of one of the tools and, optionally, a second pole line. Such embodiment of the power supply arrangement allows for providing electrical coagulation, which in some instances is beneficial or desired and, particularly, bipolar electrical coagulation, which in some instances is beneficial or desired.

Thereby, each of the plurality of tools being configured to transmit electricity from the proximal portion of one of the tools to the operative portion, when being mounted to the distal tube component is configured to either electrically connect one pole line, e.g., the first pole line or the second pole line or an additional pole line to the operative portion, or to electrically connect several pole lines, e.g., the first pole line and the second pole line to the operative portion. Like this, the tools providing electricity to their operative portions can selectively be embodied to bipolarly or mono-polarly electrification. Like this, the most appropriate electrification can be selected depending on the specific tool. Moreover, electrification can be provided by direct or alternating current.

Preferably, the tool interchange mechanism comprises a rod part having a connector tip, wherein for advancing the one of the plurality of tools from its chamber of the magazine to the distal portion of the tube component the rod part, the magazine and the tube component are configured to connect the one of the plurality of tools to the connector tip of the rod part while being arranged in one of the chambers of the magazine and to move the rod part together with the connected one of the plurality of tools through the tube component until the one of the plurality of tools is arranged at the distal portion of the tube component. Such rod part allows for an efficient advancing, withdrawal and operation of the tools.

Thereby, the tube component preferably forms the first pole line. Also, the second pole line may be arranged along the rod part of the tool interchange mechanism. Like this an efficient bi-or monopolar electrification of the operative portion of the activated tool can be achieved. More specifically, one energy pole can, e.g., be connected to the outer part of the tube component that guides the different tools to the distal portion of the tube component. The second pole can then be connected to the typically inner rod part which additionally activates the tools when they are at the distal portion of the tube component. The two energy poles can be isolated from each other through parts of the transport mechanism and/or specific isolation layers.

Thereby, the tool interchange mechanism preferably comprises a rod drive configured to move the rod part through the tube component. Such movement may particularly be a linear or translational movement and it may be performed in both directions.

The rod drive preferably comprises an engaging bar coupled to the rod part and a worm gear engaging the engaging bar or a self-locking transmission. The engaging bar can be toothed rack or a friction bar. The rod drive can further comprise a motor driving the work gear. Thereby, the motor can be provided with a bottom for being activated.

The rod part can be an essentially stable or a flexible member. In one example, the rod part is or comprises an essentially rigid rod linearly moving back and forth for activating and/or deactivating one of the tools. Moreover, it can have a flexibility in a predefined degree of freedom such as a lateral flexibility to be suitable for coiling up or the like.

Preferably, the rod part is configured to be in a collapsed position and in an expanded position, wherein in the expanded position the rod part is solid or stable along a longitudinal axis of the rod part. The term “solid along the longitudinal axis” or “stable along the longitudinal axis” relates to a configuration in which the rod part is essentially rigid when being moved back and forth. Like this, an efficient and precise activation and deactivation of a selected tool as well as an accurate operation of the activated tool is possible.

Such configuration of the rod part allows embodying the device in a compact manner. Particularly, when being retracted, i.e., after or before accessing one of the tools in the magazine, the rod part can be collapsed such that it does require comparably little space only. Like this, it can be prevented that the rod part disturbs or hinders an activity around the device. Also, the handling of the device can be improved.

In order to be collapsible, in one embodiment, the rod part can comprise rod segments configured to telescope relative to each other. For advancing the tool from the magazine to the distal portion of the tube component the rod part may expand by the rod segments sliding out of each other. In contrast, for retracting the tool from the distal portion of the tube component into the magazine, the rod part may collapse by sliding the rod segments into each other. In another embodiment, for the same purpose, the rod part may comprise an expandable and collapsible spring rod. Thereby, the tool interchange mechanism may comprise a hydraulic system configured to drive movement of the rod part.

In a particularly preferred embodiment, the rod part is rollable and in the collapsed position the rod part is rolled. Such configuration allows for a particular efficient implementation of a collapsible rod part which is embodied to be solid along the longitudinal axis of the rod part.

Thereby, the rod part preferably is displaceable about a rotational axis. Like this, it can efficiently be rolled about the rotational axis.

The rotational axis preferably is perpendicular to the longitudinal axis. This allows for a particularly compact design and efficient handling.

In a first preferred embodiment, the rod part is configured to be displaceable about the rotational axis by being bendable about the rotational axis and to be solid along the longitudinal axis by being stiff along the longitudinal axis when being unrolled. Such configuration can, e.g., be embodied by means of a tongue-like or flat bar-like rod or the like.

In a second preferred embodiment, the rod part preferably comprises a plurality of rod segments configured to be tilted relative to each other to be displaced about the rotational axis and to be solid along the longitudinal axis when being unrolled. A such segmented rod part can be efficiently rolled in order to be collapsed.

In a third preferred embodiment, the first and second embodiments are combined.

Preferably, the microinvasive surgery device comprises a tool operation force recognition structure configured to recognize at the proximal portion of the tube component an amount of a force applied for operating the activated tool mounted to the distal portion of the tube component. The term “at the proximal portion of the tube component” can particularly be in or at the body element or at a component coupled to the tube component.

Such tool operation force recognition structure allows for providing information about the haptic of operating the activated tool to the practitioner or surgeon. Like this, a specifically convenient and accurate operation is possible.

Preferably, the tool operation force recognition structure comprises an operating lever unit, wherein the operating lever unit is manually movable relative to the body element and coupled to the activated tool mounted to the distal portion of the tube component in order to operate the activated tool by moving the operating lever relative to the body element, and wherein the force applied for operating the activated tool mounted to the distal portion of the tube component correlates to a manual force applied to the operating lever for moving the operating lever relative to the body element.

The force applied for operating the tool can correlate to the manual force by corresponding or being identical to the manual force, i.e., linear correlation. Or, it can non-linearly correlate, e.g., in a gear-like fashion.

Thereby, the body element preferably has a handle member configured to be held by one hand of a practitioner, wherein the handle member and the operating lever unit are arranged such that the operating lever unit is activatable or movable relative to the handle member by the one hand of the practitioner while holding the handle member, and wherein the handle member and the selection structure of the tool interchange mechanism are arranged such that the selection structure of the tool interchange mechanism is activatable or movable relative to the handle member by the one hand of the practitioner while holding the handle member. Like this, a one-handed operation of the device can be achieved. This allows for a particularly sophisticated and convenient operation.

The tool operation force recognition structure preferably comprises a force sensor at the proximal portion of the tube component coupled to the activated tool mounted to the distal portion of the tube component to detect the force applied for operating the activated tool. Such a force sensor allows for inclusion of haptic information in an automated system. For example, in a robotic appliance the force sensor allows to evaluate the force required for operating the activated tool such that a conclusion about the haptic of the tool can be determined.

Preferably, the magazine comprises a drum rotatably mounted about a drum axis and the plurality of chambers is arranged around the drum axis. Such a drum allows to efficiently implement an efficient magazine which can efficiently be applied.

Thereby, the tube component preferably defines a tube axis parallel to the drum axis. An axes distance between the drum axis and the tube axis preferably is identical to a chamber distance between each of the chambers and the drum axis. Like this an efficient advancing or retraction of the tools into or from the tube component can be achieved.

The magazine and the tube component preferably are configured such that depending on a rotational position of the drum one of the chambers is aligned with the tube component such that the one of the plurality of tools arranged inside the aligned chamber is movable into the tube component or the one of the plurality of tools arranged inside the tube component is movable into the aligned chamber.

Thereby, the tool interchange mechanism preferably comprises a drum drive coupled to the drum of the magazine and the selection structure of the tool interchange mechanism has a switch activatable by a finger of the one hand of the practitioner. The drum drive can comprise a motor. The switch can be part of a graphical user interface of a computing device or screen.

The drum drive of the tool interchange mechanism preferably is configured to stepwise rotate the drum of the magazine about the drum axis into rotational positions in which the chambers are aligned with the tube component. Such stepwise rotation may prevent stopping rotation in an inappropriate position in which no tool can be provided out of or into one of the chambers.

Thereby, the switch of the tool interchange mechanism preferably is configured such that when being activated the drum drive stepwise rotates the drum of the magazine. Like this, automatic tool selection can efficiently be embodied.

The tool interchange mechanism preferably comprises an identification structure identifying the one of the tools and/or the chamber of the magazine aligned with the tube component. Like this, a practitioner or surgeon can efficiently identify which tool is accessible.

The drum of the magazine preferably is laterally swingable relative the handle member between a loading position in which the chambers are accessible and an interchange position in which each of the plurality of tools arranged in the chambers is activatable. Such embodiment allows for efficiently and solidly loading and de-loading the magazine with tools.

Thereby, the magazine preferably comprises a blockage configured to fix the drum in the interchange position. Such blockage allows to prevent false handling of the magazine during operation.

As an alternative to the swingable design of the drum of the magazine, it preferably is removably mounted to the handle member. Like this, the drum can efficiently be completely separated from the handle member. This allows for achieving an efficient loading of the drum.

Preferably, the microinvasive surgery device comprises a tool rotating structure configured to rotate the activated tool mounted to the distal portion of the tube component. Such tool rotating structure allows to efficiently provide the activated tool in an appropriate orientation for a specific operation.

Thereby, the handle member and the tool rotating structure preferably are arranged such that the tool rotating structure is actuatable to rotate the activated tool mounted to the distal portion of the tube component by the one hand of the practitioner while holding the handle member.

The tool rotating structure preferably comprises a wheel element arranged around the tube component, wherein the wheel element is coupled to the activated tool mounted to the distal portion of the tube component such that rotation of the wheel element by the one hand of the practitioner while holding the handle member rotates the activated tool mounted to the distal portion of the tube component. Such a wheel element allows for a convenient and accurate handling of the tool rotating structure.

Thereby, the tool rotating structure preferably comprises at least one finger rest designed at the wheel element. For example, the finger rest can be embodied as indentation shaped to receive a finger of the practitioner.

The tool rotating structure preferably comprises an orientating formation configured to prevent the activated tool mounted to the distal portion of the tube component from rotating relative to the tube component. Alternatively, the orientating formation can be configured to hold the activated tool mounted to the distal portion of the tube component in a predefined rotational position. Such orientating structures allow for assuring that the tool is accurately oriented in the or, advantageously, in plural predefined positions when being operated. Like this, the practitioner may always be aware how the activated tool exactly is oriented during operation.

Thereby, the orientating structure preferably comprises a magnet to hold the activated tool mounted to the distal portion of the tube component in the predefined rotational position.

Preferably, the operating lever unit is movable relative to the handle member from a zero position into an operation position, in which the activated tool mounted to the distal portion of the tube component is operated. The operating position can also be composed of plural sub-positions or a position range such that an operating strength of the tool may be provided by the practitioner.

The tool rotating structure preferably comprises a lock coupled to the operating lever unit and configured to prevent rotation of the activated tool mounted to the distal portion of the tube component when the operating lever unit is in the operation position.

Preferably, the tool interchange mechanism comprises a coupling member, counter coupling structures and a coupling activator, wherein the coupling member is mounted at the connector tip of the rod part, wherein each of the plurality of tools is equipped with one of the counter coupling structures, and wherein the coupling activator is arranged such that, when the rod part is distally moved towards one of the tools, the coupling member engages the counter coupling structure of the one of the tools and, when the rod part is proximally moved such that the one of the tools is located in one of the chambers of the magazine, the coupling member disengages the counter coupling structure of the one of the tools.

In another aspect, the following embodiments of microinvasive surgery devices may be beneficial:

Embodiment 1 is a microinvasive surgery device comprising: a body element; a tube component having a proximal portion and a distal portion, wherein the proximal portion of the tube component is connected to the body element; a plurality of tools each having an operative portion to contact a target; a magazine having a plurality of chambers each configured to receive one of the plurality of tools; and a tool interchange mechanism configured to activate one of the plurality of tools by advancing the one of the plurality of tools from its chamber of the magazine through the tube component to the distal portion of the tube component and mounting the forwarded tool to the distal portion of the tube component, and to deactivate the one of the plurality of tools by demounting the tool from the distal portion of the tube component and retracting the demounted one of the tools from the distal portion of the tube component through the tube component into its chamber of the magazine, wherein the tool interchange mechanism has a selection structure to select one of the plurality of tools in the magazine to be activated.

The tool interchange mechanism comprises a rod part having a connector tip, wherein for advancing the one of the plurality of tools from its chamber of the magazine to the distal portion of the tube component the rod part, the magazine and the tube component are configured to connect the one of the plurality of tools to the connector tip of the rod part while being arranged in one of the chambers of the magazine and to move the rod part together with the connected one of the plurality of tools through the tube component until the one of the plurality of tools is arranged at the distal portion of the tube component.

Embodiment 1 is characterized by the rod part of the tool interchange mechanism being configured to be in a collapsed position and in an expanded position, wherein in the expanded position the rod part is solid along a longitudinal axis of the rod part.

The configuration of embodiment 1 as well as the embodiments described in the following allow to achieve the effects and benefits of the microinvasive surgery device according to the invention and/or of specific preferred features embodied therein as described above.

Embodiment 2 is the microinvasive surgery device of embodiment 1, wherein the rod part is rollable and in the collapsed position the rod part is rolled.

Embodiment 3 is the microinvasive surgery device of embodiment 2, wherein the rod part is displaceable about a rotational axis.

Embodiment 4 is the microinvasive surgery device of embodiment 3, wherein the rotational axis is perpendicular to the longitudinal axis.

Embodiment 5 is the microinvasive surgery device of embodiment 3 or 4, wherein the rod part is configured to be displaceable about the rotational axis by being bendable about the rotational axis and to be solid along the longitudinal axis by being stiff along the longitudinal axis when being unrolled.

Embodiment 6 is the microinvasive surgery device of embodiment 3 or 4, wherein the rod part comprises a plurality of rod segments configured to be tilted relative to each other to be displaced about the rotational axis and to be solid along the longitudinal axis when being unrolled.

Embodiment 7 is the microinvasive surgery device of any one of embodiments 1 to 6, comprising a power supply arrangement configured to provide electricity to the proximal portion of one of the tools, wherein each of the plurality of tools is configured either to transmit electricity through the proximal portion of one of the tools to the operative portion or to electrically isolate the proximal portion of one of the tools from the power supply arrangement, when being mounted to the distal portion of the tube component.

Embodiment 8 is the microinvasive surgery device of any one of embodiments 1 to 7, wherein the power supply arrangement comprises a first pole line configured to provide electricity from the body element to the proximal portion of one of the tools, when being mounted to the distal portion of the tube component, and, optionally, a second pole line configured to transmit electricity from the proximal portion of one of the tools, when being mounted to the distal portion of the tube component, to the body element.

Embodiment 9 is the microinvasive surgery device of embodiment 8, wherein each of the plurality of tools being configured to transmit electricity from the proximal portion to the operative portion, when being mounted to the distal tube component is configured to either electrically connect one pole line e.g., the first pole line or the second pole line or an additional pole line to the operative portion, or to electrically connect several pole lines e.g., the first pole line and the second pole line to the operative portion.

Embodiment 10 is the microinvasive surgery device of embodiment 9, wherein the second pole line is arranged along the rod part of the tool interchange mechanism.

Embodiment 11 is the microinvasive surgery device of embodiment 10, wherein the tool interchange mechanism comprises a rod drive configured to move the rod part through the tube component.

Embodiment 12 is the microinvasive surgery device of embodiment 10 or 11, wherein the rod drive comprises an engaging bar coupled to the rod part and a worm gear engaging the engaging bar or a self-locking transmission.

Embodiment 13 is the microinvasive surgery device of any one of embodiments 1 to 12, comprising a tool operation force recognition structure configured to recognize at the proximal portion of the tube component an amount of a force applied for operating the activated tool mounted to the distal portion of the tube component.

Embodiment 14 is the microinvasive surgery device of embodiment 13, wherein the tool operation force recognition structure comprises an operating lever unit, wherein the operating lever unit is manually movable relative to the body element and coupled to the activated tool mounted to the distal portion of the tube component in order to operate the activated tool by moving the operating lever relative to the body element, and wherein the force applied for operating the activated tool mounted to the distal portion of the tube component correlates to a manual force applied to the operating lever for moving the operating lever relative to the body element.

Embodiment 15 is the microinvasive surgery device of embodiment 14, wherein the body element has a handle member configured to be held by one hand of a practitioner, wherein the handle member and the operating lever unit are arranged such that the operating lever unit is activatable or movable relative to the handle member by the one hand of the practitioner while holding the handle member, and wherein the handle member and the selection structure of the tool interchange mechanism are arranged such that the selection structure of the tool interchange mechanism is movable relative to the handle member by the one hand of the practitioner while holding the handle member.

Embodiment 16 is the microinvasive surgery device of embodiment 13, wherein the tool operation force recognition structure comprises a force sensor at the proximal portion of the tube component coupled to the activated tool mounted to the distal portion of the tube component to detect the force applied for operating the activated tool.

Embodiment 17 is the microinvasive surgery device of any one of embodiments 1 to 16, wherein the magazine comprises a drum rotatably mounted about a drum axis and the plurality of chambers is arranged around the drum axis.

Embodiment 18 is the microinvasive surgery device of embodiment 17, wherein the tube component defines a tube axis parallel to the drum axis.

Embodiment 19 is the microinvasive surgery device of embodiment 18, wherein an axes distance between the drum axis and the tube axis is identical to a chamber distance between each of the chambers and the drum axis.

Embodiment 20 is the microinvasive surgery device of any one of embodiments 17 to 19, wherein the magazine and the tube component are configured such that depending on a rotational position of the drum one of the chambers is aligned with the tube component such that the one of the plurality of tools arranged inside the aligned chamber is movable into the tube component or the one of the plurality of tools arranged inside the tube component is movable into the aligned chamber.

Embodiment 21 is the microinvasive surgery device of embodiment 20, wherein the tool interchange mechanism comprises a drum drive coupled to the drum of the magazine and the selection structure of the tool interchange mechanism has a switch activatable by a finger of the one hand of the practitioner.

Embodiment 22 is the microinvasive surgery device of embodiment 21, wherein the drum drive of the tool interchange mechanism is configured to stepwise rotate the drum of the magazine about the drum axis into rotational positions in which the chambers are aligned with the tube component.

Embodiment 23 is the microinvasive surgery device of embodiment 22, wherein the switch of the tool interchange mechanism is configured such that when being activated the drum drive stepwise rotates the drum of the magazine.

Embodiment 24 is the microinvasive surgery device of any one of embodiments 20 to 23, wherein the tool interchange mechanism comprises an identification structure identifying the one of the tools and/or the chamber of the magazine aligned with the tube component.

Embodiment 25 is the microinvasive surgery device of any one of embodiments 17 to 24, wherein the drum of the magazine is laterally swingable relative the handle member between a loading position in which the chambers are accessible and an interchange position in which each of the plurality of tools arranged in the chambers is activatable.

Embodiment 26 is the microinvasive surgery device of embodiment 25, wherein the magazine comprises a swing blockage configured to fix the drum in the interchange position.

Embodiment 27 is the microinvasive surgery device of any one of embodiments 1 to 26, comprising a tool rotating structure configured to rotate the activated tool mounted to the distal portion of the tube component.

Embodiment 28 is the microinvasive surgery device of embodiment 27, wherein the handle member and the tool rotating structure are arranged such that the tool rotating structure is actuatable to rotate the activated tool mounted to the distal portion of the tube component by the one hand of the practitioner while holding the handle member.

Embodiment 29 is the microinvasive surgery device of embodiment 27 or 28, wherein the tool rotating structure comprises a wheel element arranged around the tube component, wherein the wheel element is coupled to the activated tool mounted to the distal portion of the tube component such that rotation of the wheel element by the one hand of the practitioner while holding the handle member rotates the activated tool mounted to the distal portion of the tube component.

Embodiment 30 is the microinvasive surgery device of embodiment 29, wherein the tool rotating structure comprises at least one finger rest designed at the wheel element.

Embodiment 31 is the microinvasive surgery device of any one of embodiments 27 to 30, wherein the tool rotating structure comprises an orientating formation configured to hold the activated tool mounted to the distal portion of the tube component in a predefined rotational position.

Embodiment 32 is the microinvasive surgery device of embodiment 31, wherein the orientating structure comprises a magnet to hold the activated tool mounted to the distal portion of the tube component in the predefined rotational position.

Embodiment 33 is the microinvasive surgery device of any one of the embodiments 1 to 32, wherein the operating lever unit is movable relative to the handle member from a zero position into an operation position, in which the activated tool mounted to the distal portion of the tube component is operated.

Embodiment 34 is the microinvasive surgery device of embodiment 33, wherein the tool rotating structure comprises a lock coupled to the operating lever unit and configured to prevent rotation of the activated tool mounted to the distal portion of the tube component when the operating lever unit is in the operation position.

Embodiment 35 is the microinvasive surgery device of any one of embodiments 1 to 34, wherein the tool interchange mechanism comprises a coupling member, counter coupling structures and a coupling activator, wherein the coupling member is mounted at the connector tip of the rod part, wherein each of the plurality of tools is equipped with one of the counter coupling structures, and wherein the coupling activator is arranged such that, when the rod part is distally moved towards one of the tools, the coupling member engages the counter coupling structure of the one of the tools and, when the rod part is proximally moved such that the one of the tools is located in one of the chambers of the magazine, the coupling member disengages the counter coupling structure of the one of the tools.

Further embodiments of surgery devices are described in the following:

Embodiment 36 is a handheld microinvasive surgery device comprising: a handle member configured to be held by one hand of a practitioner; an operating lever unit; a tube component stationary connected to the handle member; a plurality of tools; a magazine having a plurality of chambers each configured to receive one of the plurality of tools; and a tool interchange mechanism configured to activate one of the plurality of tools by advancing the one of the plurality of tools from its chamber of the magazine through the tube component to a distal portion of the tube component and coupling the forwarded tool to the distal portion of the tube component, and to deactivate the one of the plurality of tools by uncoupling the mounted tool from the distal portion of the tube component and retracting the decoupled one of the tools from the distal portion of the tube component through the tube component into its chamber of the magazine. The tool interchange mechanism has a selection structure to select one of the plurality of tools in the magazine to be activated. The operating lever unit is movable relative to the handle member to operate the activated tool mounted to the distal portion of the tube component. The handle member and the operating lever unit are arranged such that the operating lever unit is activatable or movable relative to the handle member by the one hand of the practitioner while holding the handle member. Furthermore, the handle member and the selection structure of the tool interchange mechanism are arranged such that the selection structure of the tool interchange mechanism is movable relative to the handle member by the one hand of the practitioner while holding the handle member.

Embodiment 37 is the surgery device of embodiment 36, wherein the tool interchange mechanism comprises a rod part having a connector tip, wherein for advancing the one of the plurality of tools from its chamber of the magazine to the distal portion of the tube component the rod part, the magazine and the tube component are configured to connect the one of the plurality of tools to the connector tip of the rod part while being arranged in one of the chambers of the magazine and to move the rod part together with the connected one of the plurality of tools through the tube component until the one of the plurality of tools is arranged at the distal portion of the tube component.

Embodiment 38 is the surgery device of embodiment 37, wherein the rod part comprises rod segments configured to telescope relative to each other.

Embodiment 39 is the surgery device of embodiment 37, wherein the tool interchange mechanism comprises a hydraulic system configured to drive movement of the rod part.

Embodiment 40 is the surgery device of embodiment 37, wherein the rod part comprising an expandable and collapsible spring rod.

Embodiment 41 is the surgery device of any one of embodiments 37 to 40, wherein the tool interchange mechanism comprises a rod drive configured to move the rod part through the tube component.

Embodiment 42 is the surgery device of any one of embodiments 35 to 41, wherein the magazine comprises a drum rotatably mounted about a drum axis and the plurality of chambers is arranged around the drum axis.

Embodiment 43 is the surgery device of embodiment 42, wherein the tube component defines a tube axis parallel to the drum axis.

Embodiment 44 is the surgery device of embodiment 43, wherein an axes distance between the drum axis and the tube axis is identical to a chamber distance between each of the chambers and the drum axis.

Embodiment 45 is the surgery device of any one of embodiments 42 to 44, wherein the magazine and the tube component are configured such that depending on a rotational position of the drum one of the chambers is aligned with the tube component such that the one of the plurality of tools arranged inside the aligned chamber is movable into the tube component or the one of the plurality of tools arranged inside the tube component is movable into the aligned chamber.

Embodiment 46 is the surgery device of embodiment 45, wherein the tool interchange mechanism comprises a drum drive coupled to the drum of the magazine and the selection structure of the tool interchange mechanism has a switch activatable by a finger of the one hand of the practitioner.

Embodiment 47 is the surgery device of embodiment 46, wherein the drum drive of the tool interchange mechanism is configured to stepwise rotate the drum of the magazine about the drum axis into rotational positions in which the chambers are aligned with the tube component.

Embodiment 48 is the surgery device of embodiment 47, wherein the switch of the tool interchange mechanism is configured such that when being activated the drum drive stepwise rotates the drum of the magazine.

Embodiment 49 is the surgery device of any one of embodiments 45 to 48, wherein the tool interchange mechanism comprises an identification structure identifying the one of the tools and/or the chamber of the magazine aligned with the tube component.

Embodiment 50 is the surgery device of any one of embodiments 42 to 49, wherein the drum of the magazine is laterally swingable relative the handle member between a loading position in which the chambers are accessible and an interchange position in which each of the plurality of tools arranged in the chambers is activatable.

Embodiment 51 is the surgery device of embodiment 50, wherein the magazine comprises a swing blockage configured to fix the drum in the interchange position.

Embodiment 52 is the surgery device of any one of embodiments 36 to 51,comprising a tool rotating structure configured to rotate the activated tool mounted to the distal portion of the tube component.

Embodiment 53 is the surgery device of any one of embodiments 52, wherein the handle member and the tool rotating structure are arranged such that the tool rotating structure is actuatable to rotate the activated tool mounted to the distal portion of the tube component by the one hand of the practitioner while holding the handle member.

Embodiment 54 is the surgery device of embodiment 52 or 53, wherein the tool rotating structure comprises a wheel element arranged around the tube component, wherein the wheel element is coupled to the activated tool mounted to the distal portion of the tube component such that rotation of the wheel element by the one hand of the practitioner while holding the handle member rotates the activated tool mounted to the distal portion of the tube component.

Embodiment 55 is the surgery device of embodiment 54, wherein the tool rotating structure comprises at least one finger rest designed at the wheel element.

Embodiment 56 is the surgery device of any one of embodiments 52 to 55, wherein the tool rotating structure comprises an orientating formation configured to hold the activated tool mounted to the distal portion of the tube component in a predefined rotational position.

Embodiment 57 is the surgery device of embodiment 56, wherein the orientating structure comprises a magnet to hold the activated tool mounted to the distal portion of the tube component in the predefined rotational position.

Embodiment 58 is the surgery device of any one of embodiments 36 to 57, wherein the operating lever unit is movable relative to the handle member from a zero position into an operation position, in which the activated tool mounted to the distal portion

Embodiment 59 is the surgery device of embodiment 58, wherein the tool rotating structure comprises a lock coupled to the operating lever unit and configured to prevent rotation of the activated tool mounted to the distal portion of the tube component when the operating lever unit is in the operation position.

Embodiment 60 is the surgery device of any one of embodiments 36 to 59, wherein the activated tool is mounted to the distal portion of the tube component such that a force or a movement provided to the activated tool is transferred via the tube component and/or the rod part to the handle member.

### BRIEF DESCRIPTION OF THE DRAWINGS

The microinvasive surgery device according to the invention is described in more detail hereinbelow by way of exemplary embodiments and with reference to the attached drawings, in which:

FIG. 1 shows a schematic side view of a first embodiment of a microinvasive surgery device according to the invention;

FIG. 2 shows a schematic top view on the microinvasive surgery device of FIG. 1 with section A-A of FIG. 1 being cut;

FIG. 3 shows a schematic side view on the microinvasive surgery device of FIG. 1 with section B-B of FIG. 21 being cut;

FIG. 4 shows a schematic cross-sectional side view of a part of a second embodiment of a microinvasive surgery device according to the invention;

FIG. 5 shows a schematic cross-sectional side view of a first embodiment of an activated scissors tool of the microinvasive surgery device of FIG. 4; and

FIG. 6 shows a schematic cross-sectional side view of a second embodiment of an activated scissors tool of the microinvasive surgery device of FIG. 4.

### DESCRIPTION OF EMBODIMENTS

In the following description certain terms are used for reasons of convenience and are not intended to limit the invention. The terms “right”, “left”, “up”, “down”, “under” and “above” refer to directions in the figures. The terminology comprises the explicitly mentioned terms as well as their derivations and terms with a similar meaning. Also, spatially relative terms, such as “beneath”, “below”, “lower”, “above”, “upper”, “proximal”, “distal”, and the like, may be used to describe one element's or feature's relationship to another element or feature as illustrated in the figures. These spatially relative terms are intended to encompass different positions and orientations of the devices in use or operation in addition to the position and orientation shown in the figures. For example, if a device in the figures is turned over, elements described as “below” or “beneath” other elements or features would then be “above” or “over” the other elements or features. Thus, the exemplary term “below” can encompass both positions and orientations of above and below. The devices may be otherwise oriented (rotated 90 degrees or at other orientations), and the spatially relative descriptors used herein interpreted accordingly. Likewise, descriptions of movement along and around various axes include various special device positions and orientations.

To avoid repetition in the figures and the descriptions of the various aspects and illustrative embodiments, it should be understood that many features are common to many aspects and embodiments. Omission of an aspect from a description or figure does not imply that the aspect is missing from embodiments that incorporate that aspect. Instead, the aspect may have been omitted for clarity and to avoid prolix description. In this context, the following applies to the rest of this description: If, in order to clarify the drawings, a figure contains reference signs which are not explained in the directly associated part of the description, then it is referred to previous or following description sections. Further, for reason of lucidity, if in a drawing not all features of a part are provided with reference signs it is referred to other drawings showing the same part. Like numbers in two or more figures represent the same or similar elements.

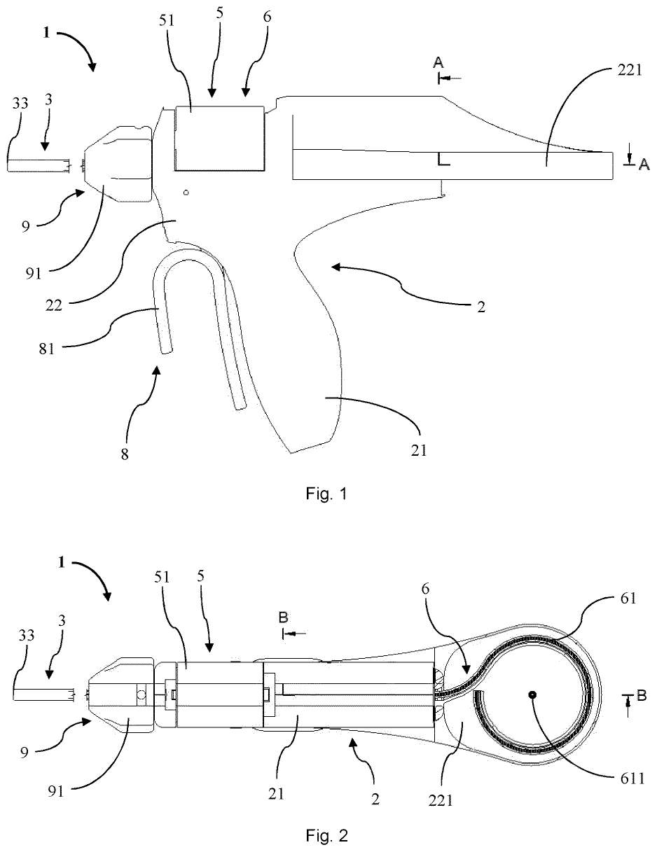

FIG. 1 shows a first embodiment of a microinvasive surgery device (MSD) 1 comprising a body element 2, a tube component 3, a magazine 5, a tool interchange mechanism 6, a tool operating force recognition structure 8 and a tool rotating structure 9. Further, the MSD 1 has a plurality of tools corresponding to the tools 40 described below in connection with a second embodiment of a MSD 10 depicted in FIGS. 4 to 6 and a power supply structure corresponding to the power supply structure 70 described below in connection with the second MSD 10 depicted in FIGS. 4 to 6.

The body element 2 has a handle member 21 configured to be held by one hand of a practitioner and a housing 22 receiving various components of the MSD 1. The housing 22 is embodied with a rod receptacle 221 designed to house a rod 61 of the tool interchange mechanism 61.

The tube component 3 has a proximal portion connected to the body element 2 and a distal portion 33. It horizontally extends from the body element 2 in a leftward direction.

The tool interchange mechanism 6 comprises a rod part 61 having a connector tip. The connector tip and the course of operation of the tool interchange mechanism 6 to activate and apply tools is similar as described in connection with the MSD 10 below. However, as can specifically be seen in FIG. 2, the rod part 61 can be arranged in a collapsed position. In particular, the rod part is bendable or displaceable about a rotational axis 611 such when being retracted, i.e., rightwardly moved relative to the tube component, e.g., by means of a motorized rod drive, it is at least partially rolled about a rotational axis 611 into the rod receptacle 211 of the housing 22 where it is safely stored in a compact manner. In the rod receptacle, the rod part 61 is in the collapsed position.

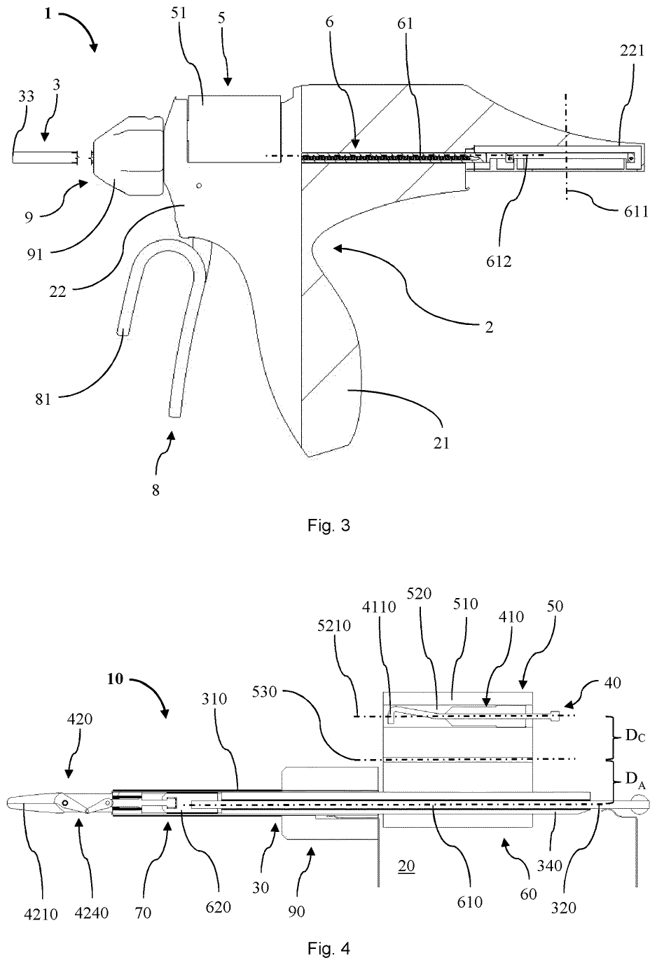

As can be best seen in FIG. 3, when being unrolled or moved into a leftward direction, which can be induced by the rod drive, the rod part 61 straightly extends along a longitudinal axis 612 into the tube component 3. In such straight arrangement, the rod part 61 is stiff along the longitudinal axis 612. Thus, in the expanded position the rod part 61 is solid along the longitudinal axis 612, which is perpendicular to the rotational axis 611.

Turning back to FIG. 1, the tool operation force recognition structure 8 comprises an operating lever unit 81. Moreover, it is configured to recognize an amount of a force applied for operating an activated tool mounted to the distal portion 33 of the tube component 3 at the lever unit 81 and, thus, also at the proximal portion of the tube component 3.

More specifically, the operating lever unit 81 is manually movable relative to the body element 2 and coupled to the activated tool mounted to the distal portion 33 of the tube component 3 in order to operate the activated tool by moving the operating lever unit 81 relative to the body element 2. The handle member 21 and the operating lever unit 81 are arranged such that the operating lever unit 81 is movable relative to the handle member 21 by the one hand of the practitioner while holding the handle member 21. Thereby, a force applied for operating the activated tool mounted to the distal portion 33 of the tube component 3 correlates to a manual force applied to the operating lever unit 81 for moving the operating lever unit 8111 relative to the body element 2.

The magazine 5 comprises a drum 51 with a plurality of chambers as described in more detail below in connection with the second MSD 10 depicted in FIGS. 4 to 7. The tool interchange mechanism 6 has a selection structure to select one of the plurality of tools in the magazine 5 to be activated. The handle member 21 and the selection structure of the tool interchange mechanism 6 are arranged such that the selection structure of the tool interchange mechanism 6 is movable relative to the handle member 21 by the one hand of the practitioner while holding the handle member 21.

The tool rotating structure 9 is configured to rotate the activated tool mounted to the distal portion 33 of the tube component 3. More specifically, the tool rotating structure 9 comprises a wheel element 91 arranged around the tube component 3. The wheel element 91 is coupled to the activated tool mounted to the distal portion 33 of the tube component 3 such that rotation of the wheel element 91 by the one hand of the practitioner while holding the handle member 21 rotates the activated tool mounted to the distal portion 33 of the tube component 3. For convenient handling, the wheel element 91 comprises plural finger rest indentations. The indentations are shaped to receive a finger of the practitioner.

The tool rotating structure 9 further comprises an orientating formation configured to prevent the activated tool mounted to the distal portion 33 of the tube component 3 from rotating relative to the tube component 3.

FIG. 4 shows some components of the second embodiment of the MSD 10 according to the invention. The MSD 10 comprises a body element 20, a tube component 30, a plurality of tools 40, a magazine 50, a tool interchange mechanism 60, a power supply structure 70 and a tool rotating structure 90. Further, the MSD 10 has a tool operating force recognition structure corresponding to the tool operating force recognition structure 8 described above in connection with the first MSD 1 depicted in FIGS. 1 to 3. Moreover, other components and configurations not shown in FIGS. 4 to 7 are similarly embodied as described above in connection with the second MSD 10 depicted in FIGS. 4 to 7.

The tube component 30 comprises a straight tube 310 receiving the rod part 610 of the tool interchange mechanism 60. The tube 310 extends into and is fixed to the body element such that a proximal portion 340 of the tube component 30 is held in the body element 20. The rod part 610 is equipped with a connector tip 620 at a left-hand or distal end.

The plurality of tools 40 comprise a hook tool 410 with a hook tip 4110 as operative portion and a first scissors tool 420 with cutting edges 4210 as operative portion and a joint formation 4240 for inducing movement of the cutting edges 4210 relative to each other. In FIG. 4, the hook tool 410 is arranged in a chamber 520 of a drum 510 of the magazine 50 and the first scissors tool 420 is activated, i.e., arranged at a distal portion 330 of the tube component 30. The first scissors tool 420 is coupled to the connector tip 620 of the rod part 610.

The drum 510 of the magazine 50 is rotatably mounted about a central drum axis 530 and the plurality of chambers 520 is regularly arranged around the drum axis 530. Each chamber 520 defines a central chamber axis 5210 extending parallel to the drum axis 530. Further, the tube component 30 defines a longitudinal tube axis 320 also parallel to the drum axis 530. An axes distance DA between the drum axis 530 and the tube axis 320 is identical to a chamber distance Dc between each of the chamber axis 5210 and the drum axis 530. Like this, depending on a rotational position of the drum 510 one of the chambers 520 is aligned with the tube component 30 such that the respective tool 40 arranged inside the aligned chamber 520 is accessible by the retracted rod part 610 and movable from the chamber 520 into the tube component 30 or vice versa.

For rotating the drum, the tool interchange mechanism 60 comprises a drum drive coupled to the drum 510 of the magazine 50 and the selection structure of the tool interchange mechanism 60 has a switch activatable by a finger of the one hand of the practitioner. The drum drive of the tool interchange mechanism 60 is configured to stepwise rotate the drum 510 about the drum axis 530 into rotational positions in which the chambers 520 are aligned with the tube component 30 and the rod part 610. The switch of the tool interchange mechanism 60 is configured such that when being activated the drum drive stepwise rotates the drum 510.

As an example of tool activation, for advancing the first scissors tool 420 from its respective chamber 520 of the magazine 50 (in FIG. 4 this is the lower chamber 520) to the distal portion 330 of the tube component 30, the respective chamber 520 is aligned with the rod part 610 and the tube 310 of the tube component 30. The magazine 50 and the tube component 30 are configured to connect the first scissors tool 420 to the connector tip 620 of the rod part 60 while being arranged the respective chamber 520 of the magazine 50 and to move the rod part 60 together with the connected scissors tool 420 through the tube 310 of the tube component 30 until the first scissors tool 420 is arranged at the distal portion 330 of the tube component 30.

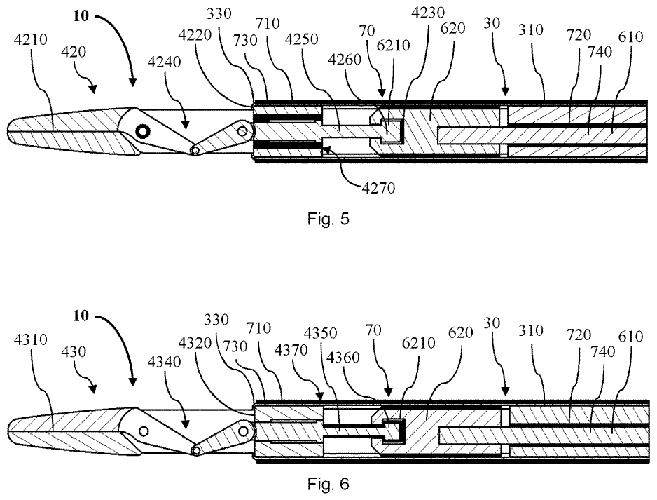

FIGS. 5 and 6 show the power supply structure 70 in more detail. In particular, the power supply structure 70 comprises the tube 310 designed as a first pole line 730, wherein the tube 310 is surrounded by an isolation 710 and a second pole line 740 arranged along the rod part 610, wherein the rod part 610 is surrounded by an isolation 720. Like this, the power supply arrangement 70 is configured to provide electricity to and from the proximal portion 4270, 4370 of the one of the tools (40). Each of the tools 40 is embodied to selectively being accessible by electricity or not.

In FIG. 5, the first scissors tool 420 is shown when being activated. In particular, it is mounted to the distal portion 330 of the tube component 30 and coupled to the rod part 610 via the connector tip 620 for operation. More specifically, the first scissors tool 420 has connection bar 4250 mounted to the joint formation 4240 at one end and coupled to the connector tip 620 at an opposite end. For coupling, the connection bar 4250 is equipped with a coupling head 4260 and the connector tip 620 with a corresponding coupling cavity 6210 receiving the coupling head 4260. Like this, axial movement of the rod part 610 relative to the tube component 30 induces opening and closing of the cutting edges 4210 of the first scissors tool 420.

The first scissors tool 420 further comprises a first connection means 4220 electrically connecting the first pole line 730 of the power supply structure 70 to the cutting edges 4210 as well as a second connection means 4230 electrically connecting the cutting edges 4210 to the second pole line 740 of the power supply structure 70. Thus, the first scissors tool 420 is configured for electrically induced bipolar coagulation.

FIG. 6 shows a second scissors tool 430 when being activated. Thereby, it is identically mounted to the distal portion 330 of the tube component 30 and coupled to the rod part 610 via the connector tip 620 for operation as the first scissors tool 420 described above. It particularly comprises identical cutting edges 4310 as operative portion, a joint formation 4340 and a connection bar 4350 with a coupling head 4360 as the first scissors tool 420.

However, for coagulation, the second scissors tool 430 has a first connection means 4320 electrically connecting the first pole line 730 of the power supply structure 70 to the proximal portion 4370 of the second scissors tool 430 but no second connection means. Rather, the proximal portion 4370 is electrically isolated from the second pole line 740 of the power supply structure 70. Thus, the second scissors tool 430 is configured for electrically induced monopolar coagulation.

Thus, as exemplified in FIGS. 5 and 6, the tools 40 of the MSD 10 are embodied to selectively electrify the operative portions as the need may be.

This description and the accompanying drawings that illustrate aspects and embodiments of the present invention should not be taken as limiting-the claims defining the protected invention. In other words, while the invention has been illustrated and described in detail in the drawings and foregoing description, such illustration and description are to be considered illustrative or exemplary and not restrictive. Various mechanical, compositional, structural, electrical, and operational changes may be made without departing from the spirit and scope of this description and the claims. In some instances, well-known circuits, structures and techniques have not been shown in detail in order not to obscure the invention. Thus, it will be understood that changes and modifications may be made by those of ordinary skill within the scope and spirit of the following claims. In particular, the present invention covers further embodiments with any combination of features from different embodiments described above and below. In particular, the components and configuration of the first and second MDS 1, 10 can be combined one single device. Thus, even though the features or structures of the MDS 1, 10 are shown in different embodiments they can be implemented in one single MDS.

The disclosure also covers all further features shown in the Figs. individually although they may not have been described in the afore or following description. Also, single alternatives of the embodiments described in the figures and the description and single alternatives of features thereof can be disclaimed from the subject matter of the invention or from disclosed subject matter. The disclosure comprises subject matter consisting of the features defined in the claims or the exemplary embodiments as well as subject matter comprising said features.

Furthermore, in the claims the word “comprising” does not exclude other elements or steps, and the indefinite article “a” or “an” does not exclude a plurality. A single unit or step may fulfil the functions of several features recited in the claims. The mere fact that certain measures are recited in mutually different dependent claims does not indicate that a combination of these measures cannot be used to advantage. The terms “essentially”, “about”, “approximately” and the like in connection with an attribute or a value particularly also define exactly the attribute or exactly the value, respectively. The term “about” in the context of a given numerate value or range refers to a value or range that is, e.g., within 20%, within 10%, within 5%, or within 2% of the given value or range. Components described as coupled or connected may be electrically or mechanically directly coupled, or they may be indirectly coupled via one or more intermediate components. Any reference signs in the claims should not be construed as limiting the scope.

1-33. (canceled)

34. A microinvasive surgery device comprising:
a body element;
a tube component having a proximal portion and a distal portion, wherein the proximal portion of the tube component is connected to the body element;
a plurality of tools each having an operative portion to contact a target, and a proximal portion;
a magazine having a plurality of chambers each configured to receive one of the plurality of tools;
a tool interchange mechanism configured to activate one of the plurality of tools by advancing the one of the plurality of tools from its chamber of the magazine through the tube component to the distal portion of the tube component and mounting the advanced tool to the distal portion of the tube component, and to deactivate the one of the plurality of tools by demounting the tool from the distal portion of the tube component and retracting the demounted one of the tools from the distal portion of the tube component through the tube component into its chamber of the magazine, wherein the tool interchange mechanism has a selection structure to select one of the plurality of tools in the magazine to be activated; and
a power supply arrangement configured to provide electricity to the proximal portion of one of the tools, when being mounted to the distal portion of the tube component,
wherein each of the plurality of tools is configured either to transmit electricity through the proximal portion to the operative portion or to electrically isolate the proximal portion of one of the tools so that no electrical transmission to the operative portion is possible, when being mounted to the distal portion of the tube component.

35. The microinvasive surgery device of claim 34, wherein the power supply arrangement comprises a first pole line configured to provide electricity from the body element to the proximal portion of one of the tools, when being mounted to the distal portion of the tube component, and, optionally, a second pole line or several pole lines configured to transmit electricity from the proximal portion of one of the tools, when being mounted to the distal portion of the tube component, to the body element.

36. The microinvasive surgery device of claim 35, wherein each of the plurality of tools being configured to transmit electricity from the proximal portion to the operative portion when being mounted to the distal tube component is configured to either electrically connect one pole line to the operative portion, or to electrically connect several pole lines to the operative portion.

37. The microinvasive surgery device of claim 34, wherein the tool interchange mechanism comprises a rod part having a connector tip, wherein for advancing the one of the plurality of tools from its chamber of the magazine to the distal portion of the tube component the rod part, the magazine and the tube component are configured to connect the one of the plurality of tools to the connector tip of the rod part while being arranged in one of the chambers of the magazine and to move the rod part together with the connected one of the plurality of tools through the tube component until the one of the plurality of tools is arranged at the distal portion of the tube component and/or wherein the tube component forms the first pole line.

38. The microinvasive surgery device of claim 37, wherein the tool interchange mechanism comprises a rod drive configured to move the rod part through the tube component.

39. The microinvasive surgery device of claim 38 wherein the rod drive comprises an engaging bar coupled to the rod part and a worm gear engaging the engaging bar or a self-locking transmission.

40. The microinvasive surgery device of any one of claim 37, wherein the rod part of the tool interchange mechanism is configured to be in a collapsed position and in an expanded position, wherein in the expanded position the rod part is solid along a longitudinal axis of the rod part.

41. The microinvasive surgery device of claim 40, wherein the rod part is rollable and in the collapsed position the rod part is rolled.

42. The microinvasive surgery device of claim 41, wherein the rod part is displaceable about a rotational axis.

43. The microinvasive surgery device of claim 42, wherein the rotational axis is perpendicular to the longitudinal axis.

44. The microinvasive surgery device of claim 42, wherein the rod part is configured to be displaceable about the rotational axis by being bendable about the rotational axis and to be solid along the longitudinal axis by being stiff along the longitudinal axis when being unrolled.

45. The microinvasive surgery device of claim 42, wherein the rod part comprises a plurality of rod segments configured to be tilted relative to each other to be displaced about the rotational axis and to be solid along the longitudinal axis when being unrolled.

46. The microinvasive surgery device of claim 34, comprising a tool operation force recognition structure configured to recognize at the proximal portion of the tube component an amount of a force applied for operating the activated tool mounted to the distal portion of the tube component and/or wherein the tool operation force recognition structure comprises an operating lever unit,
wherein the operating lever unit is manually movable relative to the body element and coupled to the activated tool mounted to the distal portion of the tube component in order to operate the activated tool by moving the operating lever unit relative to the body element, and
wherein the force applied for operating the activated tool (mounted to the distal portion of the tube component correlates to a manual force applied to the operating lever unit for moving the operating lever unit relative to the body element.

47. The microinvasive surgery device of claim 46, wherein:
the body element has a handle member configured to be held by one hand of a practitioner;
the handle member and the operating lever unit are arranged such that the operating lever unit is movable relative to the handle member by the one hand of the practitioner while holding the handle member; and
the handle member and the selection structure of the tool interchange mechanism are arranged such that the selection structure of the tool interchange mechanism is activatable by the one hand of the practitioner while holding the handle member and/or wherein the tool operation force recognition structure comprises a force sensor at the proximal portion of the tube component coupled to the activated tool mounted to the distal portion of the tube component to detect the force applied for operating the activated tool.

48. The microinvasive surgery device of claim 34, wherein the magazine comprises a drum rotatably mounted about a drum axis and the plurality of chambers is arranged around the drum axis and/or wherein the tube component defines a tube axis parallel to the drum axis and/or wherein an axes distance (D_{A}) between the drum axis and the tube axis is identical to a chamber distance (D_{C}) between each of the chambers and the drum axis.

49. The microinvasive surgery device of claims 48, wherein the magazine and the tube component are configured such that depending on a rotational position of the drum one of the chambers is aligned with the tube component such that the one of the plurality of tools arranged inside the aligned chamber is movable into the tube component or the one of the plurality of tools arranged inside the tube component is movable into the aligned chamber.

50. The microinvasive surgery device of claim 49, wherein the tool interchange mechanism comprises a drum drive coupled to the drum of the magazine and the selection structure of the tool interchange mechanism has a switch activatable by a finger of the one hand of the practitioner and/or wherein the drum drive of the tool interchange mechanism is configured to stepwise rotate the drum of the magazine about the drum axis into rotational positions in which the chambers are aligned with the tube component.

51. The microinvasive surgery device of claim 50, wherein the switch of the tool interchange mechanism is configured such that when being activated the drum drive stepwise rotates the drum of the magazine and/or wherein the tool interchange mechanism comprises an identification structure identifying the one of the tools and/or the chamber of the magazine aligned with the tube component.

52. The microinvasive surgery device of claim 50, wherein the drum of the magazine is laterally swingable relative the handle member between a loading position in which the chambers are accessible and an interchange position in which each of the plurality of tools arranged in the chambers is activatable and/or wherein the magazine comprises a swing blockage configured to fix the drum in the interchange position.

53. The microinvasive surgery device of claim 34, comprising a tool rotating structure configured to rotate the activated tool mounted to the distal portion of the tube component and/or wherein the handle member and the tool rotating structure are arranged such that the tool rotating structure is actuatable to rotate the activated tool mounted to the distal portion of the tube component by the one hand of the practitioner while holding the handle member and/or wherein the tool rotating structure comprises a wheel element arranged around the tube component, wherein the wheel element is coupled to the activated tool mounted to the distal portion of the tube component such that rotation of the wheel element by the one hand of the practitioner while holding the handle member rotates the activated tool mounted to the distal portion of the tube component and/or wherein the tool rotating structure comprises at least one finger rest designed at the wheel element and/or wherein the tool rotating structure comprises an orientating formation configured to prevent the activated tool mounted to the distal portion of the tube component from rotating relative to the tube component.
